(19) United States
(12) Patent Application Publication
(10) Pub. No.: US 2025/0339267 A1
(43) Pub. Date: Nov. 6, 2025

(54) SEALING SKIRTS FOR PROSTHETIC HEART VALVES

(71) Applicant: EDWARDS LIFESCIENCES CORPORATION, Irvine, CA (US)

(72) Inventors: Roy Shitrit, Zichron Yaakov (IL); Tamir S. Levi, Zikhron Yaakov (IL)

(73) Assignee: EDWARDS LIFESCIENCES CORPORATION, Irvine, CA (US)

(21) Appl. No.: 19/270,355

(22) Filed: Jul. 15, 2025

Related U.S. Application Data
(63) Continuation of application No. PCT/US2024/011325, filed on Jan. 12, 2024.
(60) Provisional application No. 63/480,678, filed on Jan. 19, 2023.

(51) Int. Cl. A61F 2/24; A61L 27/18

(52) U.S. Cl. CPC A61F 2/2418; A61L 27/18; A61F 2220/0075; A61F 2250/0037; A61F 2250/0069

(57) ABSTRACT

A prosthetic valve includes a frame with inflow and outflow ends and a longitudinal axis extending therebetween. The frame is in a radially expanded state defining a radially expanded diameter and is configured to define a partially radially compressed diameter when the frame is in a partially radially compressed state. A sealing skirt coupled to the frame includes inflow and outflow end portions, an inelastic skirt portion with weft and warp threads, and a tensioning element extending circumferentially along the outflow end portion. The sealing skirt includes a terminal outflow edge configured to define a skirt diameter greater or equal to the partially radially compressed diameter and less than the radially expanded diameter when the sealing skirt is in a relaxed state. The weft and warp threads are configured to be oriented at an approximately 45-degree angle to the longitudinal axis when the sealing skirt is in the relaxed state.

### CROSS REFERENCE TO RELATED APPLICATIONS

This application is a continuation of International Patent Application No. PCT/US2024/011325, filed Jan. 12, 2024, which claims the benefit of U.S. Provisional Application No. 63/480,678, filed Jan. 19, 2023. The prior applications are incorporated by reference herein in their entireties.

### FIELD

The present disclosure relates to prosthetic heart valves, and in particular to sealing skirts for prosthetic heart valves and methods for attaching a sealing skirt to a frame of a prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Most expandable, prosthetic heart valves comprise a frame or stent and a valvular structure mounted inside the frame. The frame can comprise a plurality of struts that form multiple rows of cells. Prosthetic heart valves can also include a sealing skirt coupled to the frame. The sealing skirt can be configured to assist in forming a seal between the prosthetic heart valve and a native annulus of the native valve by blocking the flow of blood through the open cells of the frame.

### SUMMARY

Described herein are prosthetic heart valves, delivery apparatus, and methods for implanting prosthetic heart valves. The disclosed prosthetic heart valves, delivery apparatus, and methods can, for example, provide for increased longevity and durability of the prosthetic heart valves. As such, the devices and methods disclosed herein can, among other things, overcome one or more of the deficiencies of typical prosthetic heart valves and their delivery apparatus.

A prosthetic heart valve can comprise a frame and a valvular structure coupled to the frame. In addition to these components, a prosthetic heart valve can further comprise one or more of the components disclosed herein.

In some examples, the frame can comprise an inflow end, an outflow end, a central longitudinal axis extending between the inflow and outflow ends of the frame, and a plurality of struts that form multiple rows of cells disposed between the inflow end and the outflow end. The frame can define a partially radially compressed diameter when the frame is in a partially radially compressed state and a radially expanded diameter when the frame is in a radially expanded state. The partially radially compressed diameter is less than the radially expanded diameter.

In some examples, the valvular structure can comprise a plurality of leaflets coupled to the inside of the frame and/or the sealing skirt.

In some examples, the prosthetic heart valve can optionally comprise a sealing skirt coupled to the inside of the frame and circumferentially disposed on an inner or an outer surface the frame.

In some examples, the sealing skirt (which can also be referred to as “a sealing member”) can be coupled to the frame and/or valvular structure and configured to reduce or prevent paravalvular leakage through and/or around the prosthetic heart valve.

In some examples, the sealing skirt can comprise an inflow end portion, an outflow end portion, and a skirt portion extending between the inflow and outflow end portions.

In some examples, the skirt portion can comprise a plurality of weft threads and a plurality of warp threads configured to be oriented at an approximately 45-degree angle relative to the central longitudinal axis of the frame when the sealing skirt is coupled to the frame and when the sealing skirt is in a relaxed or un-tensioned state.

In some examples, the outflow end portion can terminate at a terminal outflow edge. The terminal outflow edge can define a skirt diameter when the sealing skirt is in the relaxed state. The skirt diameter can optionally be greater than or equal to the partially radially compressed diameter and less than the radially expanded diameter.

In some examples, the sealing skirt can optionally comprise a tensioning element coupled to the sealing skirt and circumferentially extending along the outflow end portion.

In some examples a prosthetic heart valve can comprise a frame comprising an inflow end; an outflow end; and a central longitudinal axis extending between the inflow end and the outflow end, wherein the frame defines a radially expanded diameter when the frame is in a radially expanded state. The prosthetic heart valve can further comprise a valvular structure comprising a plurality of leaflets disposed inside the frame and a sealing skirt coupled to an inner surface of the frame. The sealing skirt can comprise an inflow end portion disposed towards the inflow end of the frame and an outflow end portion disposed towards an outflow end of the frame, wherein the outflow end portion terminates at a terminal outflow edge, the terminal outflow edge defines a skirt diameter when the sealing skirt is in a relaxed state, and the skirt diameter is less than the radially expanded diameter of the frame. The sealing skirt can further comprise a skirt portion extending between the inflow end portion and the outflow end portion, wherein the skirt portion comprises a plurality of warp threads and a plurality of weft threads oriented at an approximately 45-degree angle relative to the central longitudinal axis when the sealing skirt is in the relaxed state, and wherein the plurality of warp threads and the plurality of weft threads are formed of an inelastic polymeric material. The sealing skirt can further comprise a tensioning element stitched through the skirt portion circumferentially along the outflow end portion.

In some examples, a prosthetic heart valve can comprise a frame comprising: an inflow end; an outflow end; and a central longitudinal axis extending between the inflow end and the outflow end. The prosthetic heart valve can further comprise a sealing skirt secured to an inner surface of the frame, the sealing skirt comprising: an inflow end portion disposed towards the inflow end of the frame; an outflow end portion disposed towards the outflow end of the frame and terminating at a terminal outflow edge; a skirt portion extending between the inflow end portion and the outflow end portion, wherein the skirt portion is formed of an inelastic polymeric material; and a tensioning element coupled to and extending circumferentially along the outflow end portion.

In some examples, a prosthetic heart valve can comprise a frame comprising an inflow end and an outflow end, the frame defining a partially radially compressed diameter when the frame is in a partially radially compressed state and a radially expanded diameter when the frame is in a radially expanded state. The prosthetic heart valve can further comprise a sealing skirt coupled to an inner surface of the frame comprising an inflow end portion disposed towards the inflow end of the frame; and an outflow end portion disposed towards the outflow end of the frame terminating at a terminal outflow edge, wherein the terminal outflow edge defines a skirt diameter when the sealing skirt is in a relaxed state, the skirt diameter is greater than or equal to the partially radially compressed diameter and less than the radially expanded diameter of the frame, and the scaling skirt is formed of an inelastic polymeric material.

In some examples, a method of manufacturing a prosthetic heart valve can comprise: selecting a frame comprising an inflow end, and an outflow end, wherein the frame defines a partially radially compressed diameter in a partially radially compressed state and a radially expanded diameter in a radially expanded state; selecting a sealing skirt comprising a terminal outflow edge disposed towards an outflow end of the sealing skirt, wherein the terminal outflow edge defines a skirt diameter when the sealing skirt is in a relaxed state, and wherein the skirt diameter is greater than or equal to the partially radially compressed diameter and less than the radially expanded diameter; and coupling the sealing skirt to the frame.

In some examples, a method of manufacturing a prosthetic heart valve can comprise: tensioning a sealing skirt, wherein the sealing skirt comprises an outflow end portion disposed towards an outflow end of the prosthetic heart valve; coupling a tensioning element to the outflow end portion of the sealing skirt; and coupling the sealing skirt to a frame of the prosthetic heart valve.

In some examples, a prosthetic heart valve comprises one or more of the components recited in Examples 1-26 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of a portion of the frame of FIG. 2, showing the portion of the frame in a straightened (non-annular) state.

FIG. 4A is a cross-sectional view of a conventional prosthetic heart valve, showing a valvular structure of the conventional prosthetic heart valve in a closed state.

FIG. 4B is a cross-sectional view of the conventional prosthetic heart valve of FIG. 4A, showing the valvular structure of the conventional prosthetic heart valve in an open state.

FIG. 5A is a cross-sectional view of the prosthetic heart valve of FIG. 1, showing a valvular structure of the prosthetic heart valve in a closed state.

FIG. 5B is a cross-sectional view of the prosthetic heart valve of FIG. 1, showing the valvular structure of the prosthetic heart valve in an open state.

FIG. 6A is a side view of an outer surface of a sealing skirt for a prosthetic heart valve, according to one example, wherein the sealing skirt is shown in a flattened configuration.

FIG. 6B is a side view of an inner surface of the sealing skirt of FIG. 6A, wherein the sealing skirt is shown in a flattened configuration.

FIG. 7A is a side view of an outer surface of an sealing skirt for a prosthetic heart valve, according to a second example, wherein the sealing skirt is in a flattened configuration.

FIG. 7B is a side view of an inner surface of the sealing skirt of FIG. 7A, wherein the sealing skirt is shown in a flattened configuration.

FIG. 8 is a side view of an outer surface of a sealing skirt for a prosthetic heart valve, according to a third example, wherein the sealing skirt is in a flattened configuration.

FIG. 9 is a side view of an outer surface of a sealing skirt for a prosthetic heart valve, according to a fourth example, wherein the sealing skirt is in a flattened configuration.

FIGS. 10A-10D show a process for coupling a sealing skirt to a prosthetic heart valve, according to one example.

FIG. 11 is a side view of the prosthetic heart valve of FIG. 1, wherein the prosthetic heart valve additionally comprises an outer scaling skirt.

FIG. 12 is a side view of a prosthetic heart valve, according to a second example.

FIG. 13 is a side view of an exemplary delivery apparatus configured to deliver and implant a radially expandable prosthetic heart valve at an implantation site, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, the term “elastic” refers to an ability of a material to plastically deform and return to its original shape or configuration when forces causing the deformation are removed. As such, “elastic materials” disclosed in this application, which can include thermoplastic polyurethan (TPU), polyethylene terephthalate (PET), polyethylene (PE), ultra-high weight PE (UHMWPE), ultra-high weight PET, polytetrafluoroethylene (PTFE), etc., can have a modulus of elasticity of 0.05 GPa to 0.6 GPa, less than 1 GPa, less than 0.5 GPa, or less than 0.3 GPa. Conversely, “inelastic materials,” such as PET, PE, UHMWPE, etc., can have a modulus of elasticity in a range from of at least 0.3 GPa, at least 0.5 GPa, at least 1 GPa, or between 0.3 GPa to 3.5 GPa. However, it should be understood that the terms “inelastic” and “elastic” are comparative terms and refer to the relative elasticity between two materials in a set of materials. Thus, in any set or combination of an elastic and inelastic material referred to throughout this application and the claims, the elastic material has a lower modulus of elasticity than the inelastic material.

As used herein, the terms “free state,” “relaxed state,” and “un-tensioned state,” refer to a state of a material or component, e.g., a sealing skirt of a prosthetic heart valve, when no external forces are acting upon the material or component. This means that the material or component is not subject to external compressive or tensile forces in the free, relaxed, and un-tensioned states.

As used herein, the terms “radially expanded state” and “functional state” refer to a configuration of a prosthetic heart valve or a frame of the prosthetic heart valve in a functional configuration. In the radially expanded state, the leaflets of the prosthetic heart valve are configured to coapt with adjacent leaflets to permit blood to flow through the prosthetic heart valve from an inflow end to an outflow end of the prosthetic heart valve and prevent blood to flow through the prosthetic heart valve from the outflow end to the inflow end. Thus, the terms “radially expanded diameter,” “functional diameter,” or “functional size” refer to a nominal diameter of the prosthetic heart valve in which the prosthetic heart valve is in the radially expanded state.

As used herein, the term “radially compressed state” refers to a configuration of the prosthetic heart valve or the frame of the prosthetic heart valve in which the prosthetic heart valve is packaged in a delivery sheath of a delivery apparatus and/or inserted into the patient's vasculature on the delivery apparatus. In some examples, in which the prosthetic heart valve is crimped onto the delivery apparatus, the radially compressed state can additionally or alternatively be referred to as a “crimped state.” Thus, the terms “radially compressed diameter” and “crimped diameter” refer to a nominal diameter of the prosthetic heart valve when the prosthetic heart valve is in the radially compressed state. The radially compressed diameter is less than the expanded diameter.

As used herein, the term “partially radially compressed state” refers to a configuration of the prosthetic heart valve the frame of the prosthetic heart valve between the radially compressed state and the radially expanded state. Thus, the term “partially radially compressed diameter” refers to a nominal diameter of the prosthetic heart valve when the prosthetic heart valve is in the partially radially compressed state. The partially radially compressed diameter is less than the radially expanded diameter and greater than or equal to the radially compressed diameter.

### Overview of the Disclosed Technology

As introduced above, a prosthetic heart valve can include a frame comprising a plurality of interconnected struts, a valvular structure comprising a plurality of leaflets mounted inside the frame, and a sealing skirt circumferentially coupled to the frame and disposed between the valvular structure and an inner surface of the frame. In some examples, an outflow end portion of the sealing skirt, which terminates at a terminal outflow edge, can be disposed across the inner surface of the frame at a level of the plurality of leaflets. As such, the outflow end portion and/or the terminal outflow edge can contact the leaflets during the operation of the prosthetic heart valve. However, it has been found that reducing contact between the plurality of leaflets and other components of the prosthetic heart valve, such as the outflow end portion and the terminal outflow edge of the sealing skirt, can better increase the durability and longevity of the prosthetic heart valve.

Described herein are various inner skirts and methods for coupling inner skirts to prosthetic heart valves that help reduce contact between the inner skirt and the leaflets of the prosthetic heart valves, thereby potentially increasing the durability and longevity of the prosthetic heart valves.

### Examples of the Disclosed Technology

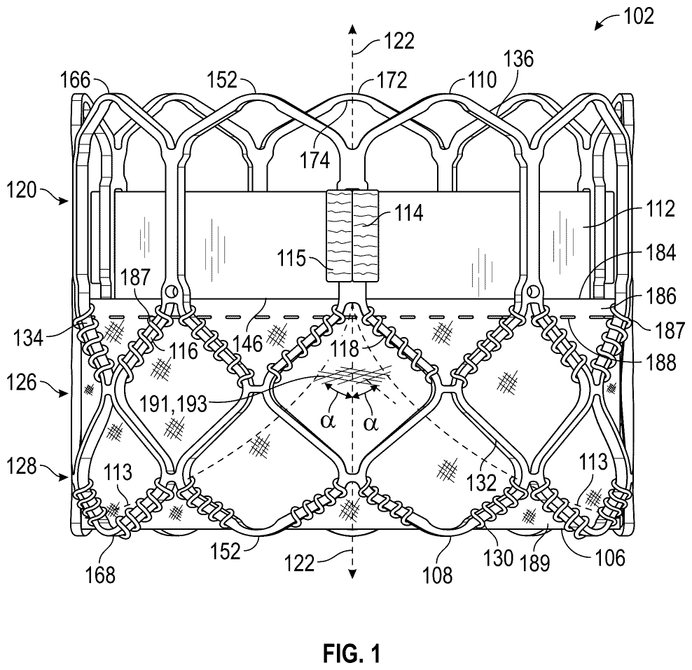

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

The prosthetic heart valve 100 can include a stent or frame 102, a valvular structure 104, and a sealing skirt 106. The prosthetic heart valve 100 (and the frame 102) can have an inflow end 108 and an outflow end 110. The valvular structure 104 can be disposed on an inside of the frame 102 and/or coupled to an inner surface of the frame 102.

In some examples, such as the example depicted in FIG. 1, the sealing skirt 106 can be disposed on the inside of the frame 102 and/or be coupled to the inner surface of the frame 102. Thus, in these examples, the sealing skirt 106 can alternatively be referred to as an “inner skirt” or an “inner sealing skirt.” As discussed below, in some examples, the valvular structure 104 can be coupled to the sealing skirt 106, which can in turn be coupled to the inner surface of the frame 102.

Although FIG. 1 depicts the sealing skirt 106 as disposed on the inside of the frame 102, other examples of sealing skirts can be disposed on the outside of the frame 102 or coupled to an outer surface of the frame 102. Thus, in these other examples, the sealing skirt 106 can alternatively be referred to as an “outer skirt” or an “outer sealing skirt.”

The valvular structure 104 can comprise a plurality of leaflets 112 (e.g., three leaflets, as shown in FIG. 1), collectively forming a leaflet structure, which can be arranged to collapse in a tricuspid arrangement. The leaflets 112 can be secured to one another at their adjacent sides (e.g., commissure tabs) to form commissures 114 of the valvular structure 104. For example, each leaflet 112 can comprise opposing commissure tabs 115 disposed on opposite sides of the leaflet 112 and a cusp edge portion extending between the opposing commissure tabs 115. The cusp edge portion of the leaflets 112 can have an undulating, curved scalloped shape.

In some examples, the leaflets 112 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

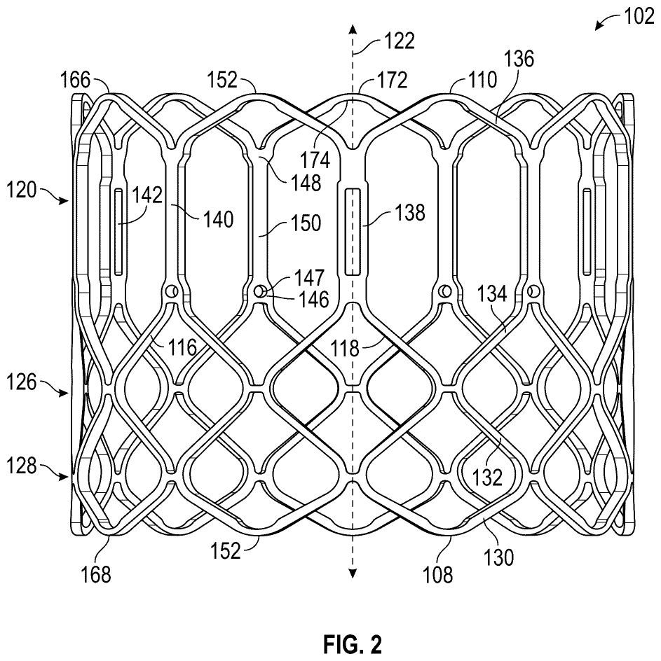

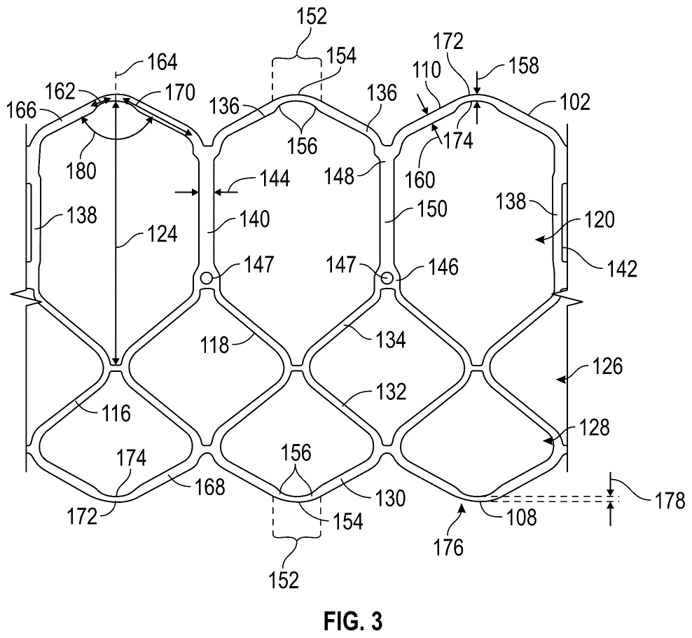

The frame 102 can be radially compressible and expandable between a radially compressed, crimped, or collapsed state and a radially expanded state (the expanded state is shown in FIG. 1). The frame 102 is shown alone in FIG. 2 and a portion of the frame 102 in a straightened (non-annular) configuration is shown in FIG. 3.

The frame 102 can be made of any of various suitable plastically-expandable materials (e.g., stainless steel, etc.) or self-expanding materials (e.g., nickel titanium alloy (NiTi), such as nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frame 102 include, without limitation, stainless steel, a nickel-based alloy (e.g., a cobalt-chromium or a nickel-cobalt-chromium alloy), polymers, or combinations thereof. In particular examples, frame 102 can be made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIGS. 2 and 3, the frame 102 can comprise a plurality of interconnected struts 116 which form multiple rows of cells 118 between the outflow end 110 and the inflow end 108 of the frame 102. In some examples, as shown in FIGS. 2 and 3, the frame 102 can comprise three rows of cells 118 with a first (upper in the orientation shown in FIGS. 2 and 3) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length 124 (FIG. 3) than cells 118 in the remaining rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (e.g., four or five) and/or more or less than nine cells per row. In some examples, the cells 118 in the first row of cells 120 may not be elongated compared to cells 118 in the remaining rows of cells of the frame 102 (the second row of cells 126 and the third row of cells 128).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of axially extending window struts 138 (or window strut portions) and a plurality of axial (or axially extending) struts 140. The axially extending window struts 138 (which can also be referred to as axial struts that include a commissure window) define commissure windows (e.g., open windows) 142 that are spaced apart from one another around the frame 102, in a circumferential direction, and which are adapted to receive a pair of commissure tabs of a pair of adjacent leaflets 112 arranged into a commissure (e.g., commissure 114 shown in FIG. 1). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (for example, two, as shown in FIGS. 2 and 3) axial struts 140 can be positioned between, in the circumferential direction, two commissure windows 142 formed by the window struts 138. Since the frame 102 can include fewer cells per row (e.g., nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 extends from a location defined by the convergence of the lower ends (e.g., ends arranged inward of and farthest away from the outflow end 110) of two angled struts 136 (which can also be referred to as an upper strut junction or upper elongated strut junction) to another location defined by the convergence of the upper ends (e.g., ends arranged closer to the outflow end 110) of two angled struts 134 (which can also be referred to as a lower strut junction or lower elongate strut junction). Each axial strut 140 and each window strut 138 forms an axial side of two adjacent cells of the first row of cells 120.

In some examples, as shown in FIG. 3, each axial strut 140 can have a width 144 (FIG. 3) that is larger than a width of the angled struts 130, 132, 134, and 136. As used herein, a “width” of a strut is measured between opposing locations on opposing surfaces of a strut that extend between the radially facing inner and outer surfaces of the strut (relative to the central longitudinal axis 122 of the frame 102). A “thickness” of a strut is measured between opposing locations on the radially facing inner and outer surfaces of a strut and is perpendicular to the width of the strut. In some examples, the width 144 of the axial struts 140 is 50-200%, 75-150%, or at least 100% larger than (e.g., double) the width of the angled struts of the frame 102.

By providing the axial struts 140 with the width 144 that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width compared to alternate prosthetic valves that have more than nine cells per row (as introduced above), the wider axial struts 140 can be more easily incorporated into the frame 102, without sacrificing open space for blood flow and/or coronary access.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts 138 forming the commissure window 142.

The cusp edge portion (e.g., scallop edge) of each leaflet 112 can be secured to the frame 102 via one or more fasteners (e.g., sutures). In some examples, the cusp edge portion of each leaflet 112 can be secured directly to the struts of the frame 102 (e.g., angled struts 130, 132, and 134). For example, the cusp edge portions of the leaflets 112 can be sutured to the angled struts 130, 132, and 134 that generally follow the contour of the cusp edge portions of the leaflets 112. Additional methods for securing the leaflets 112 to the frame 102 are disclosed in U.S. provisional patent applications 63/278,922, filed Nov. 12, 2021, and 63/300,302, filed Jan. 18, 2022, both of which are incorporated by reference herein.

In other examples, the cusp edge portion of the leaflets 112 can be secured to the scaling skirt 106, which in turn can be secured to the frame 102. For example, as shown in FIG. 1, the cusp edge portion of the leaflets 112 can be sutured to the sealing skirt 106 along a suture line 113, wherein the sealing skirt 106 is coupled to the frame 102. The leaflets 112 can be secured to the sealing skirt 106 using methods known in the art and disclosed in U.S. Pat. No. 9,393,110, which is incorporated by reference herein.

As shown in FIGS. 2 and 3, in some examples, one or more of or each of the axial struts 140 can comprise an inflow end portion 146 (e.g., an end portion that is closest to the inflow end 108) and an outflow end portion 148 that are widened relative to a middle portion 150 of the axial strut 140 (which can be defined by the width 144). In some instances, the inflow end portion 146 of the axial strut 140 can comprise an aperture 147. The apertures 147 can be configured to receive fasteners (e.g., sutures) for attaching soft components of the prosthetic heart valve 100 to the frame 102.

The interconnected struts 116 can also comprise horizontal struts 182 that extend between adjacent cells 118 of a row of cells of the frame 102 (FIGS. 2 and 3). The horizontal struts 182 can extend in a circumferential direction and also be referred to as circumferentially extending struts 182. The horizontal struts 182 can connect angled struts of two adjacent rows of angled struts of the frame 102 to one another. For example, each horizontal strut 182 can connect to two angled struts of one row of struts (for example, struts 134 shown in FIG. 3) and two angled struts in another, adjacent row of struts (for example, struts 132 shown in FIG. 3). As a result, an angled strut 183 extending between an axially extending window strut 138 and the horizontal strut 182 and an angled strut 185 extending between the horizontal strut 182 and another horizontal strut 182 disposed adjacent to the inflow end 108 of the frame 102 can be aligned along an angled line that can follow a scallop line of the leaflets (when the leaflets are attached to the frame 102). Thus, the horizontal struts 182 can allow the angled struts to follow a shape that more closely matches a shape of the scallop line of the leaflets when the frame 102 is in the radially expanded configuration (as shown in FIGS. 2 and 3). Additionally, the horizontal struts 182 can serve as spacers that can maintain a specified gap between the angled struts when the frame 102 is in the radially state configuration, thereby reducing a risk of pinching the leaflets between the struts in the radially state configuration.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Each apex region 152 can comprise an apex 154 (the highest or most outward extending, in an axial direction, point) and two thinned (or narrowed) strut portions 156, one thinned strut portion 156 extending from either side of the apex 154 to a corresponding, wider, angled strut 136 (at the outflow end 110) or angled strut 130 (at the inflow end 108) (FIG. 3). In this way, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152.

The thinned strut portions 156 of the apex regions 152 can have a width 158 that is smaller than a width 160 of the angled struts 130 or 136 (FIG. 3). In some examples, the width 158 can be a uniform width (e.g., along an entire length of the strut portion 156). In some examples, the width 158 of the thinned strut portions 156 can be from about 0.06-0.15 mm smaller than the width 160 of the angled struts 130 and/or 136.

The thinned strut portions 156 of the apex regions 152 can have a first length 162 (FIG. 3). In some examples, the first length 162 is in a range of 0.8-1.4 mm, 0.9-1.2 mm, 0.95-1.05 mm, or about 1.0 mm (e.g., ±0.03 mm). In alternate examples, the first length 162 is in a range of 0.3-0.7 mm, 0.4-0.6 mm, 0.45-0.55 mm, or about 0.5 mm (e.g., ±0.03 mm).

Thus, each outflow apex region 152 can include two thinned strut portions 156 having the first length 162, each extending from the apex 154, outward relative to a central longitudinal axis 164 of the cells 118. Thus, a total length of the apex region 152 can be two times the first length 162.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152. One half the total length of each outflow strut 166 and inflow strut 168 is shown in FIG. 3 as length 170, which extends from an end of one angled strut 136 or 130 to the central longitudinal axis 164. Thus, the length of each outflow strut 166 and inflow strut 168 is two times length 170. In some examples, the length 170 for half of each inflow strut 168 can be different than the length 170 for half of each outflow strut 166.

In some instances, the length of each thinned strut portion 156 can be at least 25% of the length 170 of the corresponding half outflow strut 166 or inflow strut 168. Said another way, the length of each apex region 152 (a total length being two times the first length 162) can be at least 25% of the total length (two times length 170) of the outflow strut 166 or inflow strut 168. In some examples, the length of each apex region 152 can be more than 25% of the total length of the corresponding outflow strut 166 or inflow strut 168, such as 25-35%.

In some examples, each apex region 152 can comprise a curved, axially facing outer surface 172 and an arcuate or curved, axially facing inner depression 174 which forms the thinned strut portions 156. For example, the curved inner depression 174 can depress toward the curved outer surface 172 from an inner surface of the angled strut portions 156, thereby forming the smaller width thinned strut portions 156. Thus, the curved inner depressions 174 can be formed on a cell side of the apex region 152 (e.g., as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152 (for example, the curved outer surface 172 can have a constant curvature).

Each apex region 152 can have a radius of curvature 176, along the curved outer surface 172 (e.g., in some instances, along an entirety or an entire length of the curved outer surface 172) (FIG. 3). In some instances, the radius of curvature 176 at the apex 154 and/or along the entire curved outer surface 172 of the apex region 152 can be greater than 1 mm. In some instances, the radius of curvature 176 can be in a range of 1-20 mm, 3-16 mm, or 8-14 mm. In some instances, the radius of curvature 176 can be greater than 10 mm. The radius of curvature 176 can be dependent on (and thus change due to changes in) the width 158 (e.g., the amount of reduction in width from the angled struts 130 or 136) and the first length 162 of the thinned strut portions 156.

Further, a height (an axial height) 178 of the apex regions 152, which can be defined in the axial direction from an outer surface of the two angled struts 130 or 136 to the curved outer surface 172 of the apex region 152 at the apex 154, can be the width 158 of the thinned strut portions 156 (FIG. 3). In this way, the height 178 of the apex regions 152 can be relatively small and not add much to the overall axial height of the radially expanded frame 102. Thus, the leaflets 112 secured to the frame 102 (FIG. 1) can be disposed closer to the inflow end 108, thereby leaving a larger open space at the outflow end 110 of the frame 102 that is not blocked by the leaflets 112.

In some examples, each of the apex region 152 can form an angle 180 between the two angled struts 130 or 136 extending from either side of the corresponding apex region 152 (FIG. 3). In some instances, the angle 180 can be in a range of 120 (not inclusive) to 140 degrees (e.g., such that the angle 180 is greater than 120 degrees and less than or equal to 140 degrees).

Additional details and examples of frames for prosthetic heart valves that include apex regions can be found in PCT Application No. PCT/US2022/025687, which is incorporated by reference herein.

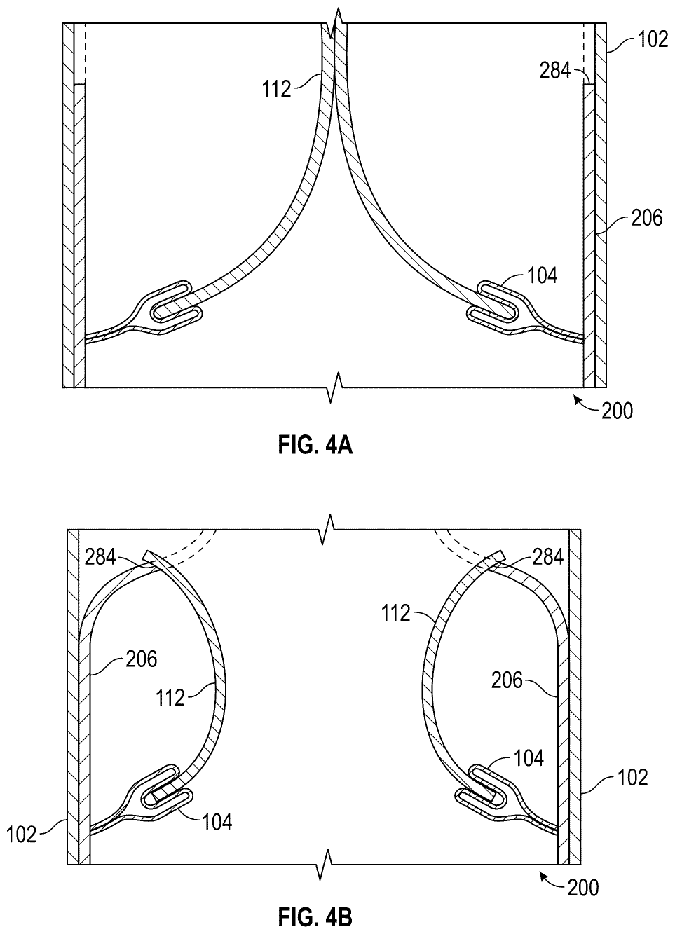

As shown in FIGS. 4A-4B, a conventional prosthetic heart valve 200 can comprise the frame 102, the valvular structure 104, and a conventional sealing skirt 206. The valvular structure 104 can be coupled to the conventional sealing skirt 206 and can comprise a plurality of leaflets 112 disposed inside the frame 102. The conventional sealing skirt 206 can be coupled to an inner surface of the frame 102 and can comprise a terminal outflow edge 284 disposed at an outflow end of the sealing skirt 206.

As shown in FIG. 4A, the conventional sealing skirt 206 of the conventional prosthetic heart valve 200 does not contact the leaflets 112 of the valvular structure 104 when the valvular structure 104 is in a closed state configured to reduce or prevent blood flow through the valvular structure 104.

As shown in FIG. 4B, the conventional sealing skirt 206 of the conventional prosthetic heart valve 200 can contact the leaflets 112 of the valvular structure 104 when the valvular structure 104 is in an open state configured to permit blood flow through the valvular structure 104. In the illustrated example, the terminal outflow edge 284 of the conventional sealing skirt 206 can contact the leaflets 112 when the conventional sealing skirt 206 collapses inwardly towards a radial center of the conventional prosthetic heart valve 200. However, as indicated by the dashed lines extending past the terminal outflow edge 284, other portions of the conventional sealing skirt 206, such as an intermediate portion of the conventional sealing skirt 206, can contact the leaflets 112 in other examples.

The conventional sealing skirt 206 is more likely to collapse radially inwards if there is excess slack in the conventional sealing skirt 206 or if the conventional sealing skirt 206 is too large relative to the frame 102. However, it can be difficult to ensure a taut fit between frame 102 and the conventional sealing skirt 206, which is formed from an inelastic material, because the inelastic material cannot be tensioned to reduce slack. Furthermore, due to the small size of prosthetic heart valves and tight tolerances involved in manufacturing sealing skirts for prosthetic heart valves, it can be difficult to fabricate a conventional sealing skirt 206 with minimal excess slack.

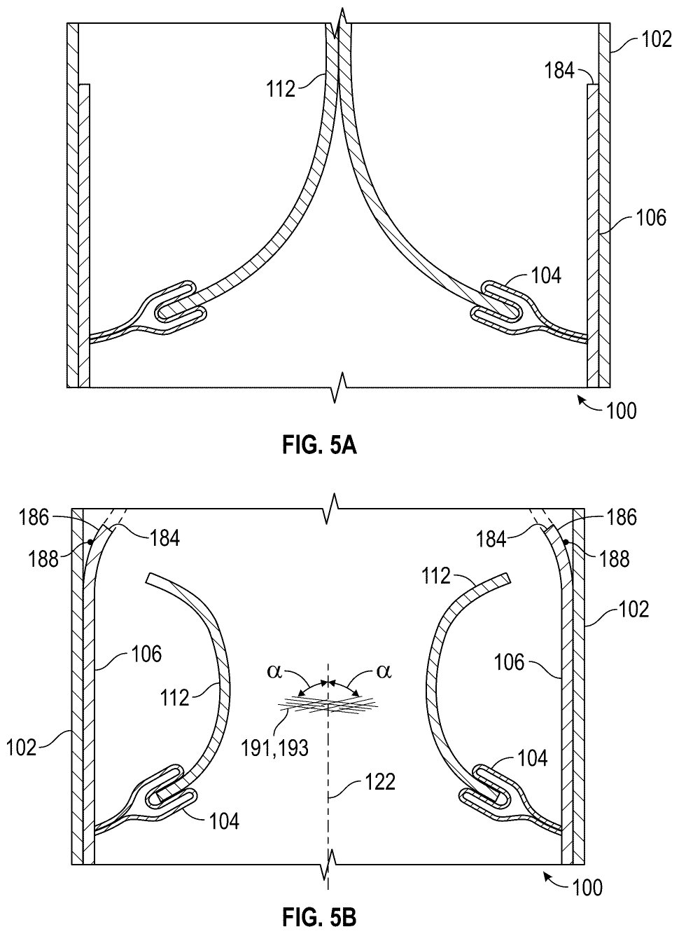

As shown in FIGS. 5A-5B, the prosthetic heart valve 100 previously shown in FIG. 1 can be configured to overcome the deficiencies of the prior art, e.g., the conventional prosthetic heart valve 200 shown in FIGS. 4A-4B. The prosthetic heart valve 100 can comprise the frame 102, the valvular structure 104 with the plurality of leaflets 112 disposed inside the frame 102, and the sealing skirt 106 disposed between the valvular structure 104 and the inner surface of the frame 102.

In other examples, not shown in FIGS. 5A-5B, the sealing skirt 106 can be disposed on the outside of the frame 102 instead of on the inside of the frame 102.

The sealing skirt 106 can comprise a terminal outflow edge 184 and an outflow end portion 186. The outflow end portion 186 can be disposed towards an outflow end of the sealing skirt 106 and can terminate at the terminal outflow edge 184. The terminal outflow edge 184 can be configured to form an annular opening at the outflow end of the sealing skirt 106 when the sealing skirt 106 is coupled to the frame 102.

In some examples, the annular opening formed by the terminal outflow edge 184 of the sealing skirt 106 can define a skirt diameter. When the sealing skirt 106 is in a free, relaxed, or un-tensioned state, the sealing skirt 106 can be optionally “undersized,” in that the skirt diameter can be greater than or equal to a radially compressed diameter of the frame 102 in a partially radially compressed state but less than a radially expanded diameter of the frame 102 in a radially expanded state. However, after the sealing skirt 106 is coupled to the frame 102 and the frame is deployed to the radially expanded state, the sealing skirt 106 can become tensioned such that the diameter of the tensioned sealing skirt 106 becomes equal to the radially expanded diameter of the frame 102.

The sealing skirt 106 can be formed of one or more skirt portions configured to be coupled together to form an annular sealing skirt. Each of the skirt portions can be formed of an inelastic polymeric material with desirable properties for sealing skirts, such as polyethylene terephthalate (PET), polyethylene (PE), ultra-high molecular weight polyethylene (UHMWPE), etc. Any of the sealing skirts disclosed in the present application can be formed of one or more of these inelastic polymeric materials.

In some examples, one or more of the skirt portions can be optionally woven from a plurality of weft threads 191 and a plurality of warp threads 193 to form an inelastic fabric. Each of the pluralities of weft threads and warp threads 191, 193 can be formed of an inelastic polymeric material, such as any of the inelastic polymeric materials disclosed in the present application.

In some examples, the skirt portions woven from the pluralities of weft and warp threads 191, 193 can have a thread count or thread density of approximately 7 threads per millimeter (e.g., ±1 thread per millimeter).

In some examples, the plurality of weft threads 191 and the plurality of warp threads 193 can be oriented at an angle (α) relative to the central longitudinal axis 122 of the frame 102 when the sealing skirt 106 is in a free, relaxed, or un-tensioned state. In some of these examples, the angle (α) can be approximately 45 degrees (e.g., ±5 degrees), but it should be understood that orienting the pluralities of weft and warp threads 191, 193 at a 45-degree angle beneficially provides the pluralities of weft and warp threads 191, 193 with the greatest ability to pivot, which therefore provides the sealing skirt 106 with the greatest ability to elongate.

However, as shown in FIG. 5B, the angle (α) between the pluralities of weft and warp threads and the central longitudinal axis 122 of the frame 102 can increase to approximately 90 degrees when the sealing skirt 106 is coupled to the frame 102 and the frame 102 is in the radially expanded state. In such circumstances, the pluralities of weft and warp threads can substantially aligned and approximately perpendicular to the central longitudinal axis 122.

As shown in FIGS. 5A-5B, when the sealing skirt 106 is coupled to the frame 102 and the frame 102 is deployed to the radially expanded state, the pluralities of weft threads and warp threads 191, 193 are configured to pivot at least slightly relative to each other such that the angle (α) increases to become a substantially perpendicular angle of approximately 90 degrees (e.g., ±5 degrees).

In some examples, the sealing skirt 106 can optionally comprise a tensioning element 188. The tensioning element 188 can be coupled to a portion of the sealing skirt 106, e.g., to the outflow end portion 186 of the sealing skirt 106. Although the tensioning element 188 is stitched through the sealing skirt 106 illustrated in FIGS. 5A-5B, other examples feature alternate methods of coupling the tensioning element 188 to the sealing skirt 106, including but not limited to embroidery, fasteners, sutures, and adhesives.

In some examples, the tensioning element 188 can be formed of an elastic material, such as thermoplastic polyurethane (TPU), PET, ultra-high molecular weight PET (UHMWPET), polytetrafluoroethylene (PTFE), polyethylene (PE), ultra-high weight PE (UHMWPE), etc., with a modulus of elasticity less than the modulus of elasticity of the inelastic material that forms the skirt portions of the sealing skirt 106. Any of the tensioning elements 188 disclosed in the present application can be formed from any of these elastic materials.

In some examples, the tensioning element 188 can comprise an elastic suture with a 4-0 (“four ought”) or 5-0 (“five ought”) diameter. In some examples, the tensioning element 188 can comprise a multi-filament elastic suture. The use of the multi-filament elastic suture can be beneficial because multi-filament elastic sutures are less abrasive than single-filament elastic sutures. However, in other examples, the tensioning element 188 can comprise a single-filament elastic suture, which can be beneficial because single-filament elastic sutures can be more clastic than multi-filament elastic sutures.

As shown in FIG. 5A, the sealing skirt 106 does not contact the frame 102 when the valvular structure 104 is in the closed state.

As shown in FIG. 5B, the sealing skirt 106 of the prosthetic heart valve 100—unlike the sealing skirt 206 of the conventional prosthetic heart valve 200 shown in FIG. 4B—is configured to minimize contact with the leaflets 112 when the valvular structure 104 is in the open state. As such, the sealing skirt 106 is shown in FIG. 5B as not contacting the leaflets 112. Thus, the sealing skirt 106 can help decrease contact between the sealing skirt 106 and the leaflets 112 by reducing excess slack in the sealing skirt 106. Such sealing skirts 106 are beneficial because, as previously discussed, reducing contact between the sealing skirt 106 and the leaflets 112 can beneficially increase the longevity and durability of the prosthetic heart valve 100.

The inventors discovered a method of coupling an “undersized” sealing skirt to a frame of a prosthetic heart valve, wherein a skirt diameter of the sealing skirt in the relaxes state is greater than or equal to the radially compressed diameter of the frame and less than the radially expanded diameter of the frame in the radially expanded state. This method of coupling sealing skirts with less excess skirt material to frames of prosthetic heart valves can beneficially reduce slack in the sealing skirts, thereby reducing unwanted contact between the scaling skirt and the leaflets.

Furthermore, the inventors also discovered that orienting a sealing skirt such that the weft threads and the warp threads are at an angle of approximately 45 degrees relative to the central longitudinal axis of a frame is beneficial. Orienting the pluralities of weft and warp threads at such an angle allows the inelastic fabric, whose threads are formed from an inelastic polymeric material, to pivot at least slightly relative to each other so that the pluralities of weft and warp threads become increasingly perpendicular to the central longitudinal axis. As such, an inelastic skirt can elongate as the frame moves from a radially expanded, axially compressed state to a radially compressed, axially elongated state despite the individual fibers not elastically deforming. This feature can beneficially enable smaller sealing skirts with less excess skirt material to be coupled to frames of prosthetic heart valves, thereby reducing slack in the sealing skirts and reducing contact between the sealing skirt and the leaflets.

Finally, the inventors discovered that it is desirable to provide a tensioning element along a portion (e.g., an outflow end portion) of an inelastic sealing skirt that tensions the sealing skirt such that a terminal outflow edge of the sealing skirt is positioned away from the leaflets (such that it does not contact the leaflets during valve operation). This feature can beneficially reduce slack in scaling skirts made of inelastic materials, thereby reducing contact between the sealing skirt and the leaflets. In this manner, the sealing skirt formed of an inelastic polymeric material and comprising one or more tensioning elements provides the advantages of sealing skirt formed of an inelastic material (e.g., frictional engagement and/or tissue ingrowth with the native tissue) and increases the life of the prosthetic leaflets.

The sealing skirts and methods described herein for coupling sealing skirts to frames of prosthetic heart valves can be applied to various other skirts, frames, prosthetic heart valves, or alternative implantable medical devices (such as stents). Additionally, the features and methods described herein can be applied to a variety of other skirts and skirt edge portions, such as other portions configured to be attached to various points of a frame (for example, the outflow end, the inflow end, or a portion of the frame disposed between the inflow and outflow end).

The skirt features and methods for coupling skirts to frames of prosthetic heart valves described herein can be similarly applied to outer sealing skirts. For example, the outer sealing skirt may contact the leaflets by collapsing radially inward between adjacent struts of the frame or through one or more cells of the frame. The features and methods disclosed herein with respect to sealing skirts can be additionally or alternatively applied to outer sealing skirts to similarly increase the longevity and durability of prosthetic heart valves.

Finally, the features illustrated and described herein-including methods of coupling “undersized” sealing skirts to frames, sealing skirts formed of pluralities of angled weft and warp threads, and tensioning elements for sealing skirts-can be implemented independently from each other or in various combinations and sub-combinations with each other. Although some of these features are illustrated or described together in the same set of figures or written descriptions for convenience and conciseness, such groupings do not preclude the possibility that these features can be implemented independently from each other in various other examples of scaling skirts for prosthetic heart valves.

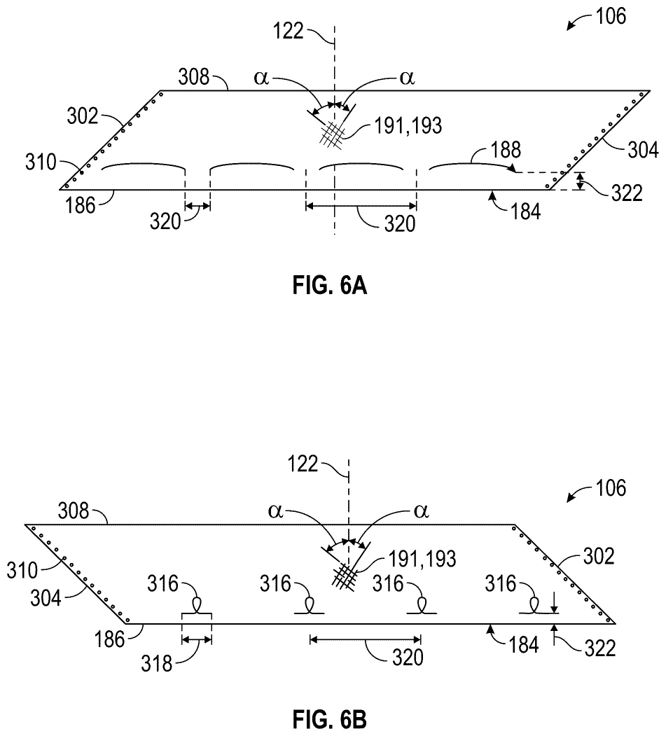

FIG. 6A illustrates a side view of an outer surface of the sealing skirt 106 for the prosthetic heart valve 100, according to one example, wherein the scaling skirt 106 is shown in a flattened configuration. The sealing skirt 106 can comprise one or more skirt portions connected together and/or individually connected to the frame 102 shown in FIG. 1 to form an annular skirt.

The skirt portions can define opposing first and second edge portions 302, 304 (which can also be referred to as short edges or edge portions) which each extend between the outflow end portion 186 and an inflow end portion 308 (which can also be referred to as long edges or edge portions) of the sealing skirt 106.

In some examples, the first and second edge portions 302, 304 can be non-perpendicular to the inflow end portion 308. For example, the first and second edge portions 302, 304 can extend at angles of approximately 45 degrees (e.g., ±5 degrees) relative to the inflow end portion 308. Therefore, an overall general shape of the scaling skirt 106 in the flattened configuration can be that of a rhomboid or parallelogram.

In some examples, the first and second edge portions 302, 304 can each comprise a plurality of apertures 310 extending therethrough. Thus, when the scaling skirt 106 is converted into an annular configuration (e.g., when mounted to the prosthetic heart valve 100 as shown in FIG. 1), the first and second edge portions 302, 304 can overlap one another with their respective apertures 310 overlapping as well. A suture can then be used to form a plurality of stitches in and in-and-out pattern through the overlapping apertures 310, thereby securing the first and second edge portions 302, 304 together and forming the annular configuration of the scaling skirt 106.

The outflow end portion 186 is an end of the sealing skirt 106 disposed closer to the outflow end of the prosthetic heart valve 100 (e.g., the outflow end 110 shown in FIG. 1) than the inflow end portion 308 when the sealing skirt 106 is coupled to the prosthetic heart valve 100. The outflow end portion 186 can terminate at the terminal outflow edge 184 that is configured to form an annular outflow opening when the sealing skirt 106 is coupled to the frame 102. In some examples, the terminal outflow edge 184 may comprise a rough, abrasive, or “molten” edge to prevent the sealing skirt 106 from fraying or unraveling.

In some examples, the outflow end portion 186 can optionally comprise the tensioning element 188 coupled to the sealing skirt 106. The tensioning element 188 can be configured to tension a portion of the sealing skirt 106, such as the terminal outflow edge 184 and/or the outflow end portion 186, between adjacent axial struts (e.g., the axial struts 140 shown in FIGS. 2-3) or adjacent angled struts (e.g., the angled struts 134 shown in FIGS. 2-3) when the sealing skirt 106 is coupled to the frame 102. The tension provided by the tensioning element 188 helps prevent the terminal outflow edge 184 from collapsing radially inwards when the sealing skirt 106 is coupled to the frame 102, thereby beneficially reducing potential contact between the sealing skirt 106 and the leaflets 112 of the prosthetic heart valve 100.

FIG. 6B illustrates a side view of an inner surface of the sealing skirt 106, wherein the scaling skirt 106 is shown in a flattened configuration. The tensioning element 188 can comprise an elastic suture stitched through the skirt portions of the sealing skirt 106 and along the outflow end portion 186. In the illustrated example, the tensioning element 188 is stitched through the sealing skirt 106 to form a plurality of whip stitches 316 in a whip stitch pattern. Each whip stitch 316 can be formed by (1) piercing a needle and the tensioning element 188 through the outer surface of the sealing skirt 106, (2) moving the needle and the tensioning element 188 along the inner surface of the sealing skirt 106 in a direction opposite the whip stitch pattern, (3) piercing the needle and the tensioning element 188 through the inner surface of the sealing skirt 106, thereby creating a loop or a bending point, and (4) running the needle and the elastic tensioning element 188 along the outer surface of the sealing skirt 106 to the next whip stitch 316. Each of the whip stitches 316 can define a whip stitch length (L₁) 318 of approximately 1.5 millimeters (e.g., ±0.5 millimeters). In the illustrated example, the whip stitch length 318 can be the diameter of the loop or bending point formed by the whip stitch 316. The pattern of whip stitches 316 can have an o.c. spacing or whip stitch pitch (L₂) 320 of approximately 2 millimeters (e.g., ±0.5 millimeters), meaning that the whip stitches 316 are spaced approximately 2 millimeters apart as measured from the geometric centers of the respective adjacent whip stitches 316. However, in other examples, the method of forming the whip stitches 316 can be mirrored such that the whip stitches 316 are formed on the outer surface of the sealing skirt 106.

In some examples, the whip stitch length (L₁) 318 and the whip stitch pitch (L₂) 320 of the whip stitches 316 can define a ratio (i.e., a ratio of L₁:L₂). In some examples, the ratio is less than one half, signifying that a greater portion or length of the tensioning element 188 runs along the outer surface of the sealing skirt 106 (i.e., the surface illustrated in FIG. 6A) than a portion or length of the tensioning element 188 running along the inner surface of the sealing skirt 106 (i.e., the surface illustrated in FIG. 6B). Lower ratios can desirably reduce the amount of the tensioning element 188 disposed on the inner surface of the sealing skirt 106, which faces the leaflets 112, thereby further reducing contact between the tensioning element 188 and the leaflets 112 of the prosthetic heart valve 100. Thus, since lower ratios signify less contact between the leaflets 112 and other components of the prosthetic heart valve 100, lower ratios can beneficially increase the longevity and durability of the prosthetic heart valve 100. In some of these examples, the ratio can be in a range from 0.2 to 0.5. In further of these examples, the ratio can be in a range from 0.3 to 0.38.

In some examples, the distance in the axial direction between the terminal outflow edge 184 and the tensioning element can define an axial distance (L₃) 322. The axial distance (L₃) 322 can be specified such that the tensioning element 188 is spaced far enough away from the terminal outflow edge 184 to prevent fraying or unraveling of the outflow end portion 186. For example, in some instances, the axial distance 322 can be approximately 2 mm (for example, ±0.5 mm).

In some examples, it can be preferable to use the whip stitches 316 to stitch the tensioning element 188 through the skirt portions of the sealing skirt 106 because the whip stitches 316 are easier to manufacture than other types of stitches. Furthermore, the process for manufacturing the whip stitches 316 can be more easily automated than processes for manufacturing other types of stitches. However, other stitching patterns, including those disclosed elsewhere throughout the present application and those known to persons with ordinary skill in the art, have other desirable properties and can be substituted for the whip stitching pattern depicted in FIGS. 6A-6B.

Although the sealing skirt 106 is not shown in FIGS. 6A-6B as being coupled to the frame 102, it should be understood that the central longitudinal axis 122 of the frame 102 can be shown for reference throughout the figures (e.g., FIGS. 6A-6B, 7A-7B, 8, 9, and 10A-10B) to better illustrate the angular orientation of the pluralities of weft and warp threads 191, 193.

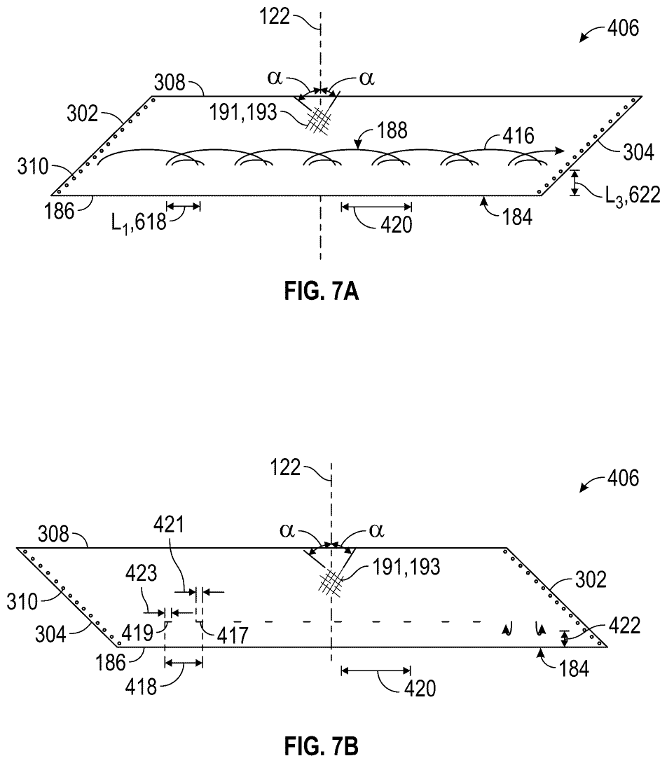

FIG. 7A illustrates a side view of an outer surface of a sealing skirt 406 for a prosthetic heart valve, according to a second example, wherein the sealing skirt 406 is in a flattened configuration. The sealing skirt 406 depicted in FIGS. 7A-7B can have various similarities and differences to the sealing skirt 106 depicted in FIGS. 1, 5A-5B, and 6A-6B.

One difference between the sealing skirt 406 and the sealing skirt 106 is that the tensioning element 188 can be stitched through the skirt portions of the sealing skirt 406 using a back stitch pattern comprising a plurality of back stitches 416. Each back stitch 416 can be formed by (1) piercing a needle and the tensioning element 188 through the outer surface of the sealing skirt 406, (2) moving the needle and tensioning element 188 along the inner surface of the sealing skirt 406 in the direction of the back stitch pattern, (3) piercing the needle and the tensioning element 188 through the inner surface of the sealing skirt 406, thereby creating a forward portion 417 of the back stitch 416, (4) running the needle and the tensioning element 188 along the outer surface of the sealing skirt 406 in a direction opposite the back stitch pattern, (5) piercing the needle and the tensioning element 188 through the outer surface of the sealing skirt 406, (6) moving the needle and tensioning element 188 along the inner surface of the sealing skirt 406 in the direction opposite the back stitch pattern, (7) piercing the needle and the tensioning element 188 through the inner surface of the sealing skirt 406, thereby creating a rearward portion 419 of the back stitch 416, and (8) running the needle and the tensioning element 188 along the outer surface of the sealing skirt 406 to the next back stitch 416.

In some examples, each back stitch 416 can define a back stitch length (L₁) 418 of approximately 1.5 millimeters (for example, ±0.5 millimeters). The pattern of back stitches 416 can have an o.c. spacing or back stitch pitch (L₂) 420 of approximately 2 millimeters (for example, ±0.5 millimeters), meaning that adjacent back stitches 416 are spaced approximately 2 millimeters apart as measured from the geometric center of the respective adjacent back stitches 416.

In some examples, the distance in the axial direction between the terminal outflow edge 184 and the tensioning element can define an axial distance (L₃) 422. The axial distance (L₃) 422 can be specified such that the tensioning element 188 is spaced far enough away from the terminal outflow edge 184 to prevent fraying or unraveling of the sealing skirt 106. For example, in some instances, the axial distance 622 can be about 2 mm (for example, ±0.1 mm). In some examples, the axial distance 422 can be equal to the axial distance 322 depicted in FIGS. 6A-6B.

In some examples, the forward portion 417 of the back stitch 416 can define a forward portion length (L₄) 421 of approximately X millimeters (for example, ±0.5 millimeters). The rear portion 419 of the back stitch 416 can define a rear portion length (L₅) 423 of approximately 2 millimeters (for example, ±0.5 millimeters).

In some examples, the sum of the forward portion length (L₄) 421 and the rear portion length (L₅) 423 and the back stitch pitch (L₂) 418 define a ratio, e.g., (L₄+L₅):L₂. In some examples, the ratio is less than one half, signifying that a greater portion or length of the tensioning element 188 runs along the outer surface of the sealing skirt 406 (i.e., the surface illustrated in FIG. 7A) than a portion or length of the tensioning element 188 running along the inner surface of the sealing skirt 406 (i.e., the surface illustrated in FIG. 7B). Lower ratios can desirably reduce the amount of the tensioning element 188 disposed on the inner surface of the sealing skirt 406, which faces the leaflets 112, thereby further reducing contact between the tensioning element 188 and the leaflets 112 of the prosthetic heart valve 100. Thus, lower ratios can beneficially increase the longevity and durability of the prosthetic heart valve 100. In some of these examples, the ratio can be in a range from 0.2 to 0.5. In further of these examples, the ratio can be in a range from 0.3 to 0.38.

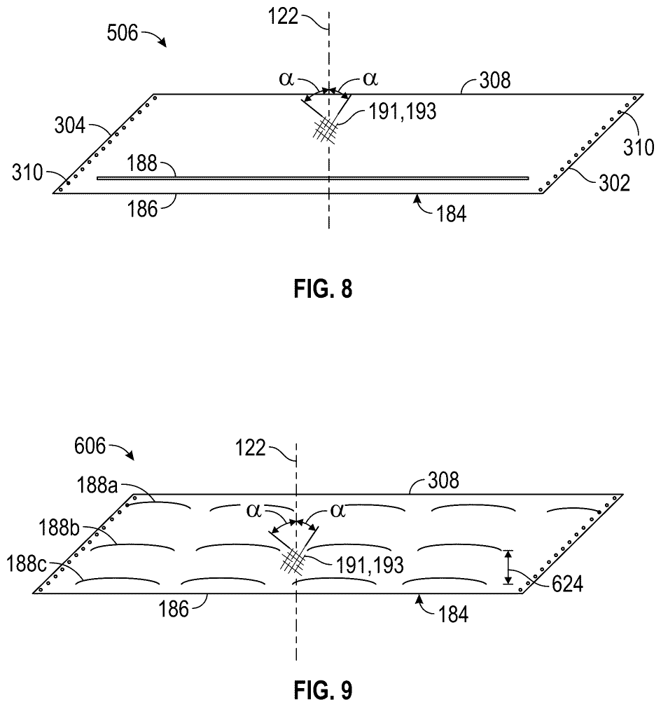

FIG. 8 illustrates a side view of an outer surface of a sealing skirt 506 for a prosthetic heart valve, according to a third example, wherein the sealing skirt 506 is in a flattened configuration. One difference between the sealing skirt 506 and the previously-described sealing skirts 106, 406 is that the tensioning element 188 can be coupled only to the outer surface of the skirt portions. Coupling the tensioning element 188 to only the outer surface of the sealing skirt 506 can beneficially prevent any portion of tensioning element 188 contacting the leaflets 112. The tensioning element 188 can be coupled to the outer surface of the sealing skirt 506 using methods including but not limited to embroidery, adhesives, and fasteners.

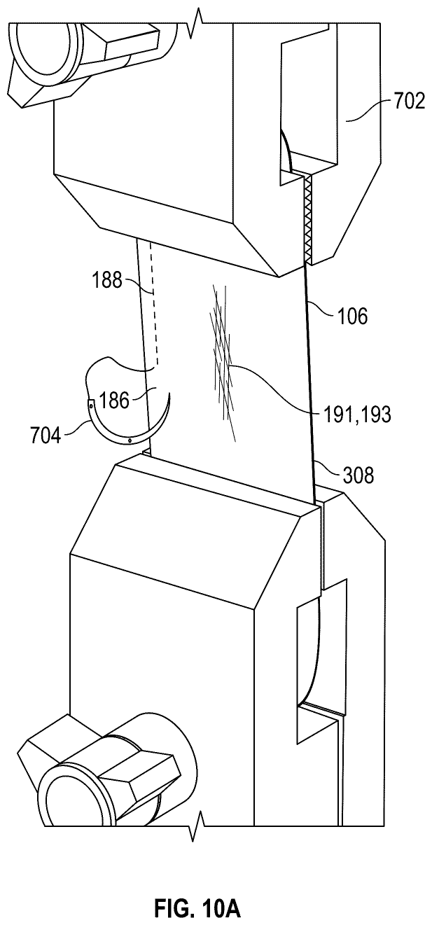

FIG. 9 illustrates a side view of an outer surface of a sealing skirt 606 for a prosthetic heart valve, according to a fourth example, wherein the sealing skirt 606 is in a flattened configuration. One difference between the sealing skirt 606 and the previously described sealing skirts 106, 406, 506 is that the sealing skirt 606 can comprise a plurality of tensioning elements 188a, 188b, 188c coupled to the skirt portions of the sealing skirt 606. The plurality of tensioning elements 188a, 188b, 188c can be disposed between the outflow end portion 186 and the inflow end portion 308. Although three tensioning elements are shown in the illustrated example, the sealing skirt 606 can have two, four, five, six, or any suitable number of tensioning elements.

In some examples, the plurality of tensioning elements 188a, 188b, 188c can be evenly spaced between the outflow end portion 186 and the inflow end portion 308 at an axial o.c. spacing or axial pitch (L₆) 624 of approximately 2 millimeters (for example, ±0.5 millimeters).

Although FIG. 9 illustrates the plurality of tensioning elements 188a, 188b, 188c stitched to the sealing skirt 606 using the whip stitch pattern illustrated in FIGS. 6A-6B, it should be understood that other stitching patterns, including those disclosed elsewhere throughout the present application and those known to persons with ordinary skill in the art, can be substituted for the whip stitch pattern illustrated in FIG. 9. Furthermore, it should be understood that other methods of coupling tensioning elements to sealing skirts, such as embroidery, mechanical fasteners, and adhesive, can be used to couple the plurality of tensioning elements 188a, 188b, 188c to the sealing skirt 606.

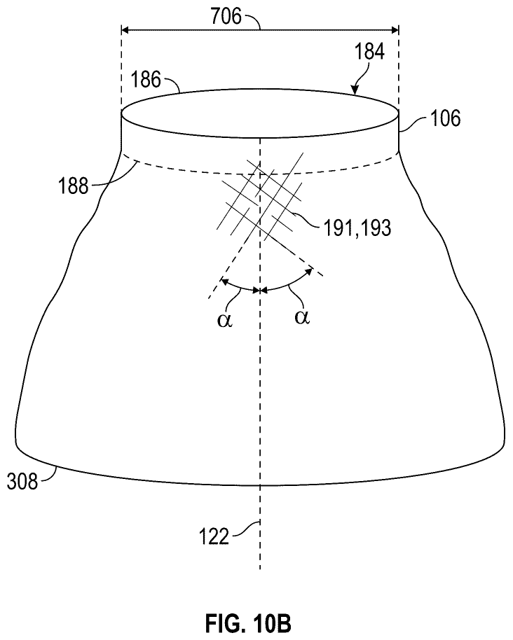

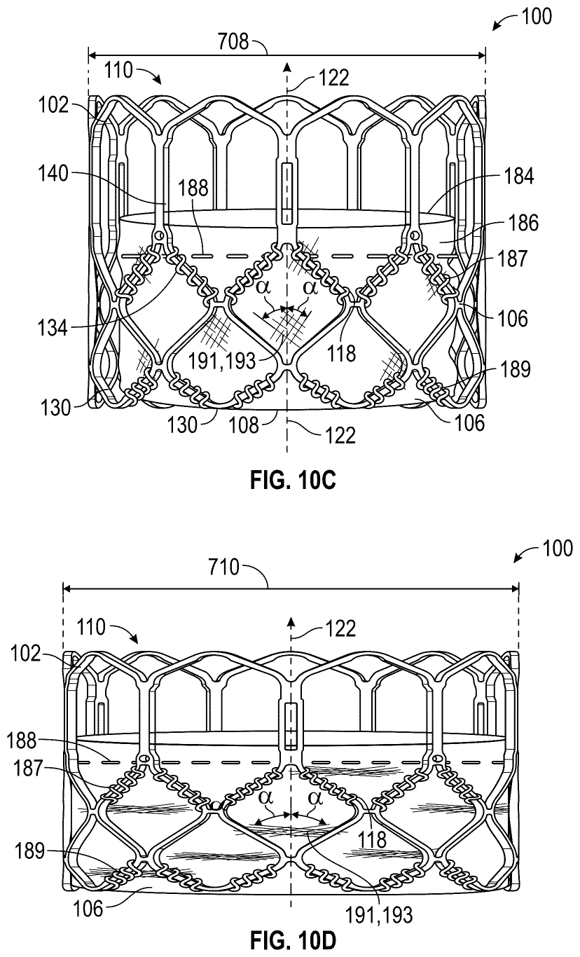

FIGS. 10A-10D show an exemplary method for coupling the sealing skirt 106 to the frame 102 of the prosthetic heart valve 100, according to one example. In other examples of this method, the prosthetic heart valve 100 can be substituted with any other of the prosthetic heart valves disclosed in the present application. Furthermore, in other examples of this method, the sealing skirt 106 illustrated in this method can be substituted with any other sealing skirt disclosed in the present application.

FIG. 10A illustrates an optional step of coupling the tensioning element 188 to the skirt portions of the sealing skirt 106. This step is optional because, as previously mentioned, some examples of the scaling skirt 106 do not comprise the tensioning element 188. The sealing skirt 106 can be loaded in a jig or a fixture 702 that is configured to tension the scaling skirt 106. While the fixture 702 is depicted as a set of two vice grips, it should be understood that any fixture suitable for tensioning the sealing skirt 106 can be used.

In examples in which the sealing skirt 106 comprises skirt portions woven from the pluralities of weft and warp threads 191, 193, tensioning the scaling skirt 106 can result in the pluralities of weft and warp threads 191, 193 pivoting relative to each other such that the pluralities of weft and warp threads 191, 193 form angles of at least 45 degrees relative to an axis extending between the outflow end portion 110 and the inflow end portion 308, i.e., the central longitudinal axis 122. In other words, the greatest angle between the plurality of weft threads 191 and the plurality of warp threads 193 can be an obtuse angle greater than 90 degrees.

Once the sealing skirt 106 is tensioned in the fixture 702, the tensioning element 188 can be coupled to the skirt portions of the sealing skirt 106, e.g., at the outflow end portion 186. In the illustrated example, the tensioning element 188 can be coupled to the sealing skirt 106 by stitching the tensioning element 188 through the skirt portions comprised of an inelastic polymeric material. The tensioning element 188 can be under tension when it being coupled to the sealing skirt 106. Although the illustrated example shows the tensioning element 188 being stitched through the skirt portions of the sealing skirt 106 using a suture needle 704, it should be understood that any stitching apparatus or method known in the art can be used to stitch the tensioning element 188 through the skirt portions of the sealing skirt 106.

FIG. 10B illustrates the sealing skirt 106 after it is removed from the fixture 702. When the sealing skirt 106 is removed from the fixture 702, the sealing skirt 106 can be in the free, relaxed, or un-tensioned state in which no external forces are acting upon it. The tensioning element 188, now no longer under tension, can contract and thus can cause the skirt portions of the sealing skirt 106 to bunch or ruffle at the outflow end portion 186 of the sealing skirt 106.

When the sealing skirt 106 is in an annular configuration and in the free, relaxed, or un-tensioned state, the terminal outflow edge 184 of the sealing skirt 106 can form an annular opening defining a skirt diameter 706. Although the sealing skirt 106 is shown in the annular configuration before being coupled to the frame 102 to better illustrate the skirt diameter 706, the sealing skirt 106 in some examples does not assume the annular configuration until after it is coupled to the frame 102.

In examples in which the sealing skirt 106 comprises skirt portions woven from the pluralities of weft and warp threads 191, 193, the pluralities of weft and warp threads 191, 193 can pivot relative to each other in the free, relaxed, or un-tensioned state such that the pluralities of weft and warp threads 191, 193 can form angles of approximately 45 degrees relative to the axis extending between the outflow end portion 186 and the inflow end portion 308, e.g., central longitudinal axis 122. In other words, the plurality of weft threads 191 and the plurality of warp threads 193 can form angles relative to each other of approximately 90 degrees.

FIG. 10C shows the sealing skirt 106 being coupled to the frame 102, wherein the frame 102 can be a partially radially compressed state. The partially radially compressed state can be between a radially compressed state and a radially expanded state. In the partially radially compressed state, the frame 102 defines a partially radially compressed diameter 708, which is the nominal diameter of the frame 102 in the partially radially compressed state. The partially radially compressed diameter 708 can be less than or equal to the skirt diameter 706.

The sealing skirt 106 can be coupled to the frame 102 using sutures, fasteners, or any other known methods of coupling sealing skirts to frames. In some examples, such as the example illustrated in FIG. 10C, the sealing skirt 106 can be secured to the angled struts 134 of the frame 102 using a first set of sutures 187. Additionally or alternatively, the sealing skirt 106 can be secured to the axial struts 140. The sealing skirt 106 can be additionally or alternatively be secured to the frame 102 at the inflow end 108 by suturing the sealing skirt 106 to the first row of angled struts 130 using a second set of sutures 189. Additional methods of coupling skirts to frames are disclosed in U.S. Pat. No. 9,393,110.

FIG. 10D shows the prosthetic heart valve 100, which includes the frame 102 and the sealing skirt 106, being deployed to the radially expanded state, which can also be referred to as a “functional state.” In some examples, the prosthetic heart valve 100 may be deployed to the radially expanded state during surgery, i.e., after the prosthetic heart valve 100 is compressed or crimped onto a delivery apparatus, delivered through a patient's vasculature, and positioned in the native annulus. While in the radially expanded state, the leaflets 112 of the prosthetic heart valve 100 are configured to coapt with adjacent leaflets 112 to permit blood to flow through the prosthetic heart valve 100 from the inflow end 108 to the outflow end 110 of the prosthetic heart valve 100 and prevent blood flow through the prosthetic heart valve 100 from the outflow end 110 to the inflow end 108.

Although the prosthetic heart valve 100 can include additional components, such as the valvular structure 104, these additional components are omitted in FIG. 10D to more clearly show the sealing skirt 106.

When the frame 102 is in the radially expanded state, the frame 102 defines a radially expanded diameter 710. The radially expanded diameter 710, which can be alternatively referred to as the expanded working diameter, the functional diameter, or the functional size, can be the nominal diameter of the frame 102 and/or the prosthetic heart valve 100 in the radially expanded state. The radially expanded diameter 710 can range from 20 millimeters to 29 millimeters. In some examples, the radially expanded diameter 710 is one of 20 millimeters, 23 millimeters, 26 millimeters, and 29 millimeters.

The radially expanded diameter 710 that can be greater than the skirt diameter 706 and the partially radially compressed diameter 708. Furthermore, the radially expanded diameter 710 can be greater than a radially compressed diameter of the frame 102 in a radially compressed state. In some examples, the skirt diameter 706 can be defined as a percentage of the radially expanded diameter 710. In some of these examples, the skirt diameter 706 is 70.9% to 93.5% of the radially expanded diameter 710. In other examples, the skirt diameter 706 can be a particular length less than the radially expanded diameter 710, e.g., 1.5 millimeters less than the radially expanded diameter 710.

In examples in which the sealing skirt 106 comprises skirt portions woven from the pluralities of weft and warp threads 191, 193, deploying the prosthetic heart valve 100 to the radially expanded state can result in the pluralities of weft and warp threads 191, 193 pivoting relative to each other such that the pluralities of weft and warp threads 191, 193 become substantially perpendicular to the central longitudinal axis 122. In other words, the plurality of weft threads 191 and the plurality of warp threads 193 can become substantially aligned in a direction perpendicular to the central longitudinal axis 122.

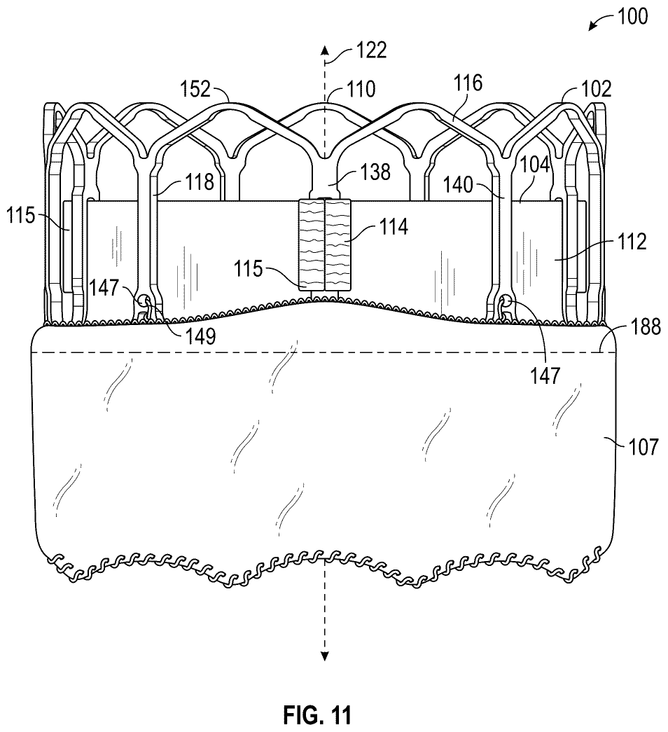

FIG. 11 illustrates the prosthetic heart valve 100 of FIG. 1 with an outer sealing skirt 107 disposed around an outer surface of the frame 102. In some examples, the outer skirt 107 can be an annular skirt configured to reduce paravalvular leakage (PVL). In some examples, the outer sealing skirt 107 can comprise one or more skirt portions that are connected together and/or individually connected to the frame 102. The outer sealing skirt 107 can comprise a fabric or polymeric material, such as expanded polytetrafluoroethylene (ePTFE), PTFE, PET, TPU, UHMWPE, PEEK, PE, etc. In some instances, instead of having a relatively straight upper edge portion, as shown in FIG. 11, the outer sealing skirt 107 can have an undulating upper edge portion that extends along and is secured to the angled struts 134. Examples of such outer sealing skirts and various other outer sealing skirts that can be used with the frame 102 can be found in U.S. provisional patent application No. 63/366,599 filed Jun. 17, 2022, which is incorporated by reference herein.

In FIG. 11, the sealing skirt 106 is hidden from view by the outer sealing skirt 107. However, the tensioning element 188 disposed on the sealing skirt 106 and behind the outer sealing skirt 107 is indicated by the dashed line. Other illustrations in the present application, such as FIG. 1, more clearly illustrate the tensioning element 188 disposed on the sealing skirt 106.

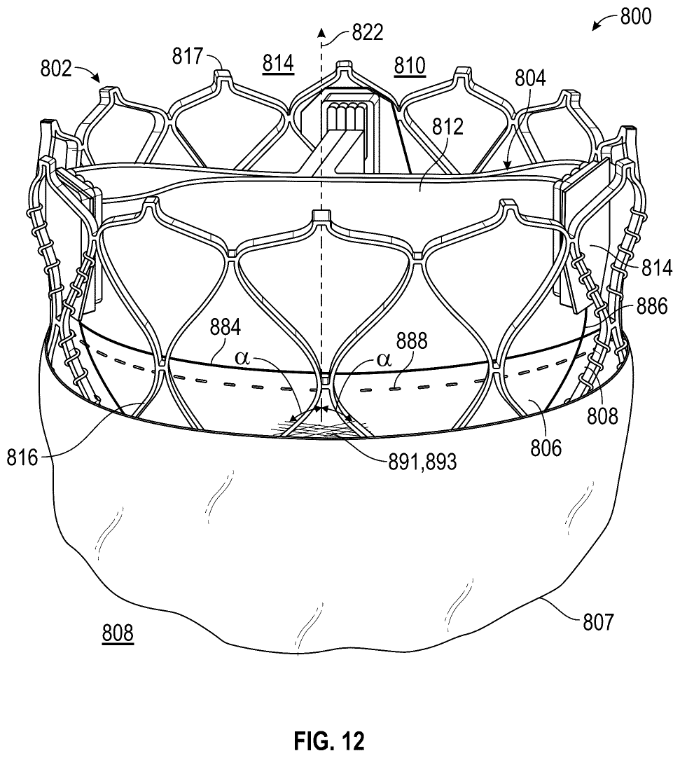

FIG. 12 shows an exemplary prosthetic heart valve 800, according to another example. The prosthetic heart valve 800 comprises four main components: a stent or frame 802, a valvular structure 804, a sealing skirt 806, and a perivalvular outer sealing member or outer sealing skirt 807. The prosthetic heart valve 800 can comprise an inflow end portion 808, an outflow end portion 810, and a central longitudinal axis 822 formed between the inflow and outflow end portions 808, 810. The sealing skirt 806 can be arranged on and/or coupled to an inner surface of the frame 802, while the outer scaling skirt 807 can be arranged on and/or coupled to an outer surface of the frame 802.

The frame 802 can be radially compressible (collapsible) and expandable and comprise a plurality of interconnected struts 816. A plurality of apices 817 that are spaced circumferentially apart are formed at the inflow end portion 808 and the outflow end portion 810 of the frame 802 (only the apices 817 at the outflow end portion 810 are visible in FIG. 11). Each apex 817 is formed at a junction between two angled struts 816 at either the inflow end portion 808 or the outflow end portion 810. FIG. 1 depicts apices 817 that form a U-shaped bend between the two angled struts 816.

The sealing skirt 806 can comprise one or more skirt portions arranged in an annular configuration. In some examples, the skirt portions can be woven from a plurality of inelastic weft and warp threads 891, 893 configured to be oriented at an angle (α) of approximately 45 degrees relative to the central longitudinal axis 822 when the sealing skirt 806 is coupled to the frame and when the scaling skirt 806 is in a free relaxed. The angle (α) is configured to increase, e.g., to an approximately 90-degree angle, when the prosthetic heart valve 800 is deployed to the radially expanded state.

The sealing skirt 806 can further comprise a tensioning element 888 extending circumferentially along an outflow end portion 886 adjacent a circumferential free edge 884 disposed at the outflow end of sealing skirt 806. The tensioning element 888 can be formed from an elastic material with a modulus of elasticity less than the modulus of elasticity of the inelastic polymeric material that forms the skirt portions of the sealing skirt 806. Delivery Apparatus

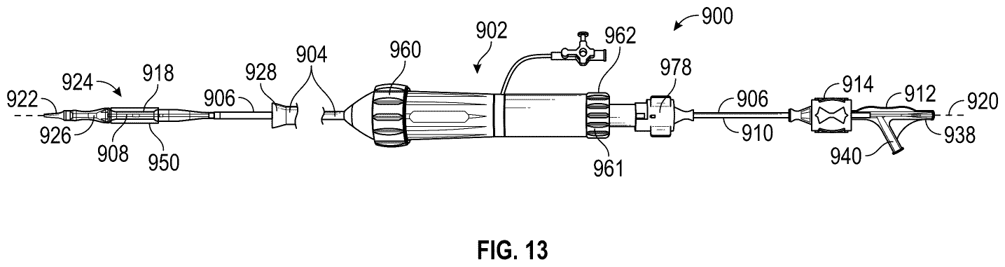

FIG. 13 shows a delivery apparatus 900, according to one example, that can be used to implant an expandable prosthetic heart valve (e.g., the prosthetic heart valve 100 of FIG. 1 and/or any of the other prosthetic heart valves described herein). In some examples, the delivery apparatus 900 is specifically adapted for use in introducing a prosthetic valve into a heart.

The delivery apparatus 900 in the illustrated example of FIG. 13 is a balloon catheter comprising a handle 902 and a steerable, outer shaft 904 extending distally from the handle 902. The delivery apparatus 900 can further comprise an intermediate shaft 906 (which also may be referred to as a balloon shaft) that extends proximally from the handle 902 and distally from the handle 902, the portion extending distally from the handle 902 also extending coaxially through the outer shaft 904. Additionally, the delivery apparatus 900 can further comprise an inner shaft 908 extending distally from the handle 902 coaxially through the intermediate shaft 906 and the outer shaft 904 and proximally from the handle 902 coaxially through the intermediate shaft 906.

The outer shaft 904 and the intermediate shaft 906 can be configured to translate (e.g., move) longitudinally, along a central longitudinal axis 920 of the delivery apparatus 900, relative to one another to facilitate delivery and positioning of a prosthetic valve at an implantation site in a patient's body.

The intermediate shaft 906 can include a proximal end portion 910 that extends proximally from a proximal end of the handle 902, to an adaptor 912. A rotatable knob 914 can be mounted on the proximal end portion 910 and can be configured to rotate the intermediate shaft 906 around the central longitudinal axis 920 and relative to the outer shaft 904.

The adaptor 912 can include a first port 938 configured to receive a guidewire therethrough and a second port 940 configured to receive fluid (e.g., inflation fluid) from a fluid source. The second port 940 can be fluidly coupled to an inner lumen of the intermediate shaft 906.

The intermediate shaft 906 can further include a distal end portion that extends distally beyond a distal end of the outer shaft 904 when a distal end of the outer shaft 904 is positioned away from an inflatable balloon 918 of the delivery apparatus 900. A distal end portion of the inner shaft 908 can extend distally beyond the distal end portion of the intermediate shaft 906.

The balloon 918 can be coupled to the distal end portion of the intermediate shaft 906.

In some examples, a distal end of the balloon 918 can be coupled to a distal end of the delivery apparatus 900, such as to a nose cone 922 (as shown in FIG. 13), or to an alternate component at the distal end of the delivery apparatus 900 (e.g., a distal shoulder). An intermediate portion of the balloon 918 can overlay a valve mounting portion 924 of a distal end portion of the delivery apparatus 900 and a distal end portion of the balloon 918 can overly a distal shoulder 926 of the delivery apparatus 900. The valve mounting portion 924 and the intermediate portion of the balloon 918 can be configured to receive a prosthetic heart valve in a radially compressed state. For example, as shown schematically in FIG. 13, a prosthetic heart valve 950 (which can be one of the prosthetic valves described herein) can be mounted around the balloon 918, at the valve mounting portion 924 of the delivery apparatus 900.

The balloon shoulder assembly, including the distal shoulder 926, is configured to maintain the prosthetic heart valve 950 (or other medical device) at a fixed position on the balloon 918 during delivery through the patient's vasculature.

The outer shaft 904 can include a distal tip portion 928 mounted on its distal end. The outer shaft 904 and the intermediate shaft 906 can be translated axially relative to one another to position the distal tip portion 928 adjacent to a proximal end of the valve mounting portion 924, when the prosthetic valve 950 is mounted in the radially compressed state on the valve mounting portion 924 (as shown in FIG. 13) and during delivery of the prosthetic valve to the target implantation site. As such, the distal tip portion 928 can be configured to resist movement of the prosthetic valve 950 relative to the balloon 918 proximally, in the axial direction, relative to the balloon 918, when the distal tip portion 928 is arranged adjacent to a proximal side of the valve mounting portion 924.

An annular space can be defined between an outer surface of the inner shaft 908 and an inner surface of the intermediate shaft 906 and can be configured to receive fluid from a fluid source via the second port 940 of the adaptor 912. The annular space can be fluidly coupled to a fluid passageway formed between the outer surface of the distal end portion of the inner shaft 908 and an inner surface of the balloon 918. As such, fluid from the fluid source can flow to the fluid passageway from the annular space to inflate the balloon 918 and radially expand and deploy the prosthetic valve 950.

An inner lumen of the inner shaft can be configured to receive a guidewire therethrough, for navigating the distal end portion of the delivery apparatus 900 to the target implantation site.

The handle 902 can include a steering mechanism configured to adjust the curvature of the distal end portion of the delivery apparatus 900. In the illustrated example, for example, the handle 902 includes an adjustment member, such as the illustrated rotatable knob 960, which in turn is operatively coupled to the proximal end portion of a pull wire. The pull wire can extend distally from the handle 902 through the outer shaft 904 and has a distal end portion affixed to the outer shaft 904 at or near the distal end of the outer shaft 904. Rotating the knob 960 can increase or decrease the tension in the pull wire, thereby adjusting the curvature of the distal end portion of the delivery apparatus 900. Further details on steering or flex mechanisms for the delivery apparatus can be found in U.S. Pat. No. 9,339,384, which is incorporated by reference herein.

The handle 902 can further include an adjustment mechanism 961 including an adjustment member, such as the illustrated rotatable knob 962, and an associated locking mechanism including another adjustment member, configured as a rotatable knob 978. The adjustment mechanism 961 is configured to adjust the axial position of the intermediate shaft 906 relative to the outer shaft 904 (e.g., for fine positioning at the implantation site). Further details on the delivery apparatus 900 can be found in PCT Application No. PCT/US2021/047056, which is incorporated by reference herein.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parastemal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium. and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the systems, devices, apparatuses, etc. herein can be sterilized (e.g., with heat, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a frame comprising: an inflow end; an outflow end; and a central longitudinal axis extending between the inflow end and the outflow end, wherein the frame defines a radially expanded diameter when the frame is in a radially expanded state; a valvular structure comprising a plurality of leaflets disposed inside the frame; and a sealing skirt coupled to an inner surface of the frame, the sealing skirt comprising: an inflow end portion disposed towards the inflow end of the frame; an outflow end portion disposed towards an outflow end of the frame, wherein: the outflow end portion terminates at a terminal outflow edge, the terminal outflow edge defines a skirt diameter when the sealing skirt is in a relaxed state, and the skirt diameter is less than the radially expanded diameter of the frame; a skirt portion extending between the inflow end portion and the outflow end portion, wherein the skirt portion comprises a plurality of warp threads and a plurality of weft threads oriented at an approximately 45-degree angle relative to the central longitudinal axis when the sealing skirt is in the relaxed state, wherein the plurality of warp threads and the plurality of weft threads are formed of an inelastic polymeric material; and a tensioning element stitched through the skirt portion circumferentially along the outflow end portion.

Example 2. A prosthetic heart valve comprising: a frame comprising: an inflow end; an outflow end; and a central longitudinal axis extending between the inflow end and the outflow end; a sealing skirt secured to an inner surface of the frame, the sealing skirt comprising: an inflow end portion disposed towards the inflow end of the frame; an outflow end portion disposed towards the outflow end of the frame and terminating at a terminal outflow edge; a skirt portion extending between the inflow end portion and the outflow end portion, wherein the skirt portion is formed of an inelastic polymeric material; and a tensioning element coupled to and extending circumferentially along the outflow end portion.

Example 3. The prosthetic heart valve of any example herein, particularly example 2, wherein the tensioning element comprises an elastic suture configured to be stitched through the skirt portion.

Example 4. The prosthetic heart valve of any example herein, particularly example 3, wherein the tensioning element is an elastic suture formed from one of TPU, PET, ultra-high weight PET, and PTFE.

Example 5. The prosthetic heart valve of any example herein, particularly example 3, wherein the tensioning element comprises one of a 4-0 (“four ought”) elastic suture and a 5-0 (“five ought”) elastic suture.

Example 6. The prosthetic heart valve of any example herein, particularly example 3, wherein the tensioning element comprises a multi-filament elastic suture.

Example 7. The prosthetic heart valve of any example herein, particularly example 3, wherein the tensioning element defines a plurality of whip stitches in the skirt portion, wherein the plurality of whip stitches is disposed circumferentially along the outflow end portion.

Example 8. The prosthetic heart valve of any example herein, particularly example 7, wherein a pitch between adjacent ones of the plurality of whip stitches ranges from 2 millimeters to 2.5 millimeters.

Example 9. The prosthetic heart valve of any example herein, particularly example 7, wherein a length of each of the plurality of whip stitches ranges from 1 millimeter to 2 millimeters.

Example 10. The prosthetic heart valve of any example herein, particularly example 3, wherein the tensioning element defines a plurality of back stitches in the skirt portion.

Example 11. The prosthetic heart valve of any example herein, particularly example 2, wherein the skirt portion is formed from one of PET, PE, and UHMWPE.

Example 12. The prosthetic heart valve of any example herein, particularly example 2, wherein the skirt portion comprises a fabric comprising pluralities of weft and warp threads formed of an inelastic polymeric material, and wherein the skirt portion has a thread count of approximately 7 yarns per millimeter.

Example 13. The prosthetic heart valve of any example herein, particularly example 2, wherein the skirt portion comprises a plurality of weft threads and a plurality of warp threads, wherein the plurality of weft threads and the plurality of warp threads are formed of an inelastic material and are configured to form an approximately 45-degree angle with the central longitudinal axis of the frame when the sealing skirt is in a relaxed state.

Example 14. The prosthetic heart valve of any example herein, particularly example 2, wherein a first length of the tensioning element is disposed on an inner surface of the skirt portion and a second length of the tensioning element is disposed on an outer surface of the skirt portion.

Example 15. The prosthetic heart valve of any example herein, particularly example 14, wherein the second length of the tensioning element disposed on the outer surface of the skirt portion is greater than the first length of the tensioning element disposed on the inner surface of the skirt portion.

Example 16. The prosthetic heart valve of any example herein, particularly example 15, wherein a ratio of the first length to the second length is in a range from 0.6 to 0.75.

Example 17. A prosthetic heart valve comprising: a frame comprising an inflow end and an outflow end, the frame defining a partially radially compressed diameter when the frame is in a partially radially compressed state and a radially expanded diameter when the frame is in a radially expanded state; and a sealing skirt coupled to an inner surface of the frame comprising: an inflow end portion disposed towards the inflow end of the frame; and an outflow end portion disposed towards the outflow end of the frame terminating at a terminal outflow edge, wherein: the terminal outflow edge defines a skirt diameter when the sealing skirt is in a relaxed state, the skirt diameter is greater than or equal to the partially radially compressed diameter and less than the radially expanded diameter of the frame, and the sealing skirt is formed of an inelastic polymeric material.

Example 18. The prosthetic heart valve of any example herein, particularly example 17, wherein the sealing skirt is formed of a plurality of inelastic warp threads and a plurality of inelastic weft threads.

Example 19. The prosthetic heart valve of any example herein, particularly example 18, wherein the frame defines a central longitudinal axis extending between the inflow end and the outflow end of the frame, and wherein each of the plurality of inelastic warp threads and each of the plurality of inelastic weft threads are oriented at an approximately 45-degree angle relative to the central longitudinal axis when the sealing skirt is in the relaxed state.

Example 20. The prosthetic heart valve of any example herein, particularly example 19, wherein the skirt diameter is 70.9% to 93.5% of the radially expanded diameter.

Example 21. A method of manufacturing a prosthetic heart valve comprising: selecting a frame comprising: an inflow end; and an outflow end, wherein the frame defines a partially radially compressed diameter in a partially radially compressed state and a radially expanded diameter in a radially expanded state; selecting a sealing skirt comprising: a terminal outflow edge disposed towards an outflow end of the sealing skirt, wherein the terminal outflow edge defines a skirt diameter when the sealing skirt is in a relaxed state, and wherein the skirt diameter is greater than or equal to the partially radially compressed diameter and less than the radially expanded diameter; and coupling the sealing skirt to the frame.

Example 22. The method of any example herein, particularly example 21, wherein the method further comprises coupling a tensioning element to the sealing skirt prior to coupling the sealing skirt to the frame.

Example 23. A method of manufacturing a prosthetic heart valve comprising: tensioning a sealing skirt, wherein the sealing skirt comprises an outflow end portion disposed towards an outflow end of the prosthetic heart valve; coupling a tensioning element to the outflow end portion of the sealing skirt; and coupling the sealing skirt to a frame of the prosthetic heart valve.

Example 24. The method of any example herein, particularly example 23, wherein the sealing skirt is tensioned by mounting the sealing skirt to a fixture.

Example 25. The method of any example herein, particularly example 23, wherein the tensioning element is coupled to the sealing skirt by stitching the tensioning element through the sealing skirt along the outflow end portion.

Example 26. A prosthetic heart valve of any example herein, particularly any one of examples 1-25, wherein the prosthetic heart valve is sterilized.

The features described herein with regard to any example can be combined with other features described in any one or more of the other examples, unless otherwise stated. For example, any one or more of the features of one skirt can be combined with any one or more features of another skirt. As another example, any one or more features of one prosthetic heart valve or device can be combined with any one or more features of another prosthetic heart valve or device.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic heart valve comprising:
a frame in a radially expanded state comprising:
an inflow end;
an outflow end; and
a central longitudinal axis extending between the inflow end and the outflow end,
wherein the frame in the radially expanded state defines a radially expanded diameter;
a valvular structure comprising a plurality of leaflets disposed inside the frame; and
a sealing skirt in a tensioned state coupled to an inner surface of the frame, the sealing skirt comprising:
an inflow end portion disposed towards the inflow end of the frame;
an outflow end portion disposed towards an outflow end of the frame, wherein:
the outflow end portion terminates at a terminal outflow edge,
the terminal outflow edge is configured to define a skirt diameter when the sealing skirt is in a relaxed state, and
the skirt diameter is less than the radially expanded diameter of the frame;
a skirt portion extending between the inflow end portion and the outflow end portion, wherein the skirt portion comprises a plurality of warp threads and a plurality of weft threads configured to be oriented at an approximately 45-degree angle relative to the central longitudinal axis when the sealing skirt is in the relaxed state, and wherein the plurality of warp threads and the plurality of weft threads are formed of an inelastic polymeric material; and
a tensioning element stitched through the skirt portion circumferentially along the outflow end portion.

2. A prosthetic heart valve comprising:
a frame comprising:
an inflow end;
an outflow end; and
a central longitudinal axis extending between the inflow end and the outflow end;
a sealing skirt secured to an inner surface of the frame, the sealing skirt comprising:
an inflow end portion disposed towards the inflow end of the frame;
an outflow end portion disposed towards the outflow end of the frame and terminating at a terminal outflow edge;
a skirt portion extending between the inflow end portion and the outflow end portion, wherein the skirt portion is formed of an inelastic polymeric material; and
a tensioning element coupled to and extending circumferentially along the outflow end portion.

3. The prosthetic heart valve of claim 2, wherein the tensioning element comprises an elastic suture configured to be stitched through the skirt portion.

4. The prosthetic heart valve of claim 3, wherein the tensioning element is an elastic suture formed from one of TPU, PET, ultra-high weight PET, and PTFE.

5. The prosthetic heart valve of claim 3, wherein the tensioning element comprises one of a 4-0 (“four ought”) elastic suture and a 5-0 (“five ought”) elastic suture.

6. The prosthetic heart valve of claim 3, wherein the tensioning element comprises a multi-filament elastic suture.

7. The prosthetic heart valve of claim 3, wherein the tensioning element defines a plurality of whip stitches in the skirt portion, wherein the plurality of whip stitches is disposed circumferentially along the outflow end portion.

8. The prosthetic heart valve of claim 7, wherein a pitch between adjacent ones of the plurality of whip stitches ranges from 2 millimeters to 2.5 millimeters.

9. The prosthetic heart valve of claim 7, wherein a length of each of the plurality of whip stitches ranges from 1 millimeter to 2 millimeters.

10. The prosthetic heart valve of claim 3, wherein the tensioning element defines a plurality of back stitches in the skirt portion.

11. The prosthetic heart valve of claim 2, wherein the skirt portion is formed from one of PET, PE, and UHMWPE.

12. The prosthetic heart valve of claim 2, wherein the skirt portion comprises a fabric comprising pluralities of weft and warp threads formed of an inelastic polymeric material, and wherein the skirt portion has a thread count of approximately 7 yarns per millimeter.

13. The prosthetic heart valve of claim 2, wherein the skirt portion comprises a plurality of weft threads and a plurality of warp threads, wherein the plurality of weft threads and the plurality of warp threads are formed of an inelastic material and are configured to form an approximately 45-degree angle with the central longitudinal axis of the frame when the sealing skirt is in a relaxed state.

14. The prosthetic heart valve of claim 2, wherein a first length of the tensioning element is disposed on an inner surface of the skirt portion and a second length of the tensioning element is disposed on an outer surface of the skirt portion.

15. The prosthetic heart valve of claim 14, wherein the second length of the tensioning element disposed on the outer surface of the skirt portion is greater than the first length of the tensioning element disposed on the inner surface of the skirt portion.

16. The prosthetic heart valve of claim 15, wherein a ratio of the first length to the second length is in a range from 0.6 to 0.75.

17. A prosthetic heart valve comprising:
a frame in a partially radially compressed state comprising an inflow end and an outflow end, the frame defining a partially radially compressed diameter and configured to define a radially expanded diameter when the frame is in a radially expanded state; and
a sealing skirt in a relaxed state coupled to an inner surface of the frame and comprising:
an inflow end portion disposed towards the inflow end of the frame; and
an outflow end portion disposed towards the outflow end of the frame terminating at a terminal outflow edge, wherein:
the terminal outflow edge of the sealing skirt in the relaxed state defines a skirt diameter,
the skirt diameter is greater than or equal to the partially radially compressed diameter and less than the radially expanded diameter of the frame, and
the sealing skirt is formed of an inelastic polymeric material.

18. The prosthetic heart valve of claim 17, wherein the sealing skirt is formed of a plurality of inelastic warp threads and a plurality of inelastic weft threads.

19. The prosthetic heart valve of claim 18, wherein the frame defines a central longitudinal axis extending between the inflow end and the outflow end of the frame, and wherein each of the plurality of inelastic warp threads and each of the plurality of inelastic weft threads of the sealing skirt in the relaxed state are oriented at an approximately 45-degree angle relative to the central longitudinal axis.

20. The prosthetic heart valve of claim 19, wherein the skirt diameter is 70.9% to 93.5% of the radially expanded diameter.
